(19) United States
(12) Patent Application Publication
(10) Pub. No.: US 2004/0127880 A1
(43) Pub. Date: Jul. 1, 2004

(54) Absorbent article with suspended absorbent pad structure

(75) Inventor: Shirlee A. Weber, Neenah, WI (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC.

(21) Appl. No.: 10/331,951

(22) Filed: Dec. 30, 2002

(51) Int. Cl. A61F 13/15; A61F 13/20

(52) U.S. Cl. 604/385.22

(57) ABSTRACT

An absorbent article includes a chassis having a front waist region with a front waistband, a back waist region with a back waistband, and a crotch region extending longitudinally between the front and back waist regions. An absorbent pad structure is sandwiched between an outer cover member and a bodyside liner. A suspension member is disposed at each longitudinal end of the absorbent pad structure, the suspension members having one end fusably bonded with a fusable thermoplastic material within the absorbent pad structure, and an opposite end attached to a respective waistband of the chassis. Upon the article being worn by a user, the absorbent pad structure is slideably disposed against the outer cover member such that the absorbent article is form-fitting without the absorbent pad structure impeding stretchability of the outer cover member.

### TECHNICAL FIELD OF THE INVENTION

The present invention relates generally to the field of absorbent articles and garments, such as children's training pants, disposable diapers, feminine hygiene articles, incontinence articles, and the like, which incorporate absorbent pad structures for absorbing and containing bodily fluids.

### BACKGROUND

Many types of consumer products such as disposable diapers, training pants, feminine care articles, incontinence articles, and the like, utilize an absorbent pad structure for absorbing and wicking away bodily fluids. The absorbent pads are conventionally formed from an absorbent web, typically a fibrous web material formed by known techniques, and are disposed between a liquid pervious bodyside liner and a liquid impermeable outer cover. The absorbent web material may be encased in a tissue wrap or facing and backing sheets. The absorbent pad structures are typically adhered or bonded to the outer cover, and may also be adhered to the bodyside liner.

There has been a general appreciation in the art that benefits may be realized by suspending the absorbent pad structure within the article chassis instead of attaching the pad to the outer cover and/or bodyside liner. For example, it has been suggested to suspend an absorbent insert between the waist sections of an absorbent article to allow unrestricted stretchability of the outer cover and form-fitting functionality of the absorbent pad structure. Reference is made, for example, to U.S. Pat. Nos. 4,808,176; 4,701,174; 4,756,709; and 6,168,585 B1. These references, however, describe relatively complicated and material intensive configurations for supporting the absorbent structures.

U.S. Pat. No. 5,389,095 describes an absorbent article having an absorbent member suspended with elastic members from an outer cover in a “hammock-like” manner so that the absorbent member does not contact the outer cover. This arrangement requires the absorbent member to have its own liquid permeable facing sheet and liquid impermeable backing sheet. Also, the “hammock-like” suspension of the absorbent member with respect to the outer cover may tend to present a bulky and ill-fitting appearance.

The present invention provides an absorbent article incorporating a relatively simple and effective system for suspending the absorbent pad structure.

### SUMMARY OF THE INVENTION

Objects and advantages of the invention will be set forth in part in the following description, or may be obvious from the description, or may be learned through practice of the invention.

In general, the present invention provides an improved absorbent article having an absorbent pad structure sandwiched between an outer cover member and a suitable bodyside liner. A particularly useful embodiment of the invention is a child's training pant. However, other absorbent articles such as incontinence devices, feminine hygiene products, disposable diapers, and the like, are also within the scope and spirit of the invention.

In one embodiment, the article has a chassis including an elastomeric outer cover member. The absorbent pad structure may be suspended on the article chassis by at least one suspension member having one end attached directly to absorbent core material of the pad structure, and an opposite end attached generally at the waistband structure of the chassis, for example between the outer cover member and a bodyside liner. The absorbent pad structure may be suspended at both longitudinal ends by a pair of the suspension members.

The pad structure includes an absorbent core material having a fuseable thermoplastic material that bonds with the suspension member in a suitable bonding process, such as thermal or ultrasonic bonding. For example, the absorbent core material may be a coform material comprising an airlaid blend of cellulosic wood fibers and meltblown polymer fibers. In one particular embodiment, the polymer fibers are elastic and the coform material is an elastic coform.

The suspension members may be elastomeric or non-elastomeric. In an embodiment wherein the suspension members are non-elastomeric, the absorbent core material may be an elastic coform such that the suspended absorbent pad structure is stretchable between its suspension points by virtue of the elastic properties of the coform material. In an alternate embodiment wherein the coform material is generally non-elastic, the suspension members may be elastomeric such that the suspended absorbent pad structure is elastically supported between its suspension points. The suspension members may be formed of or include a thermoplastic material that also softens and bonds with the absorbent core material in a suitable bonding process. For example, the suspension members may be a nonwoven web of polymer fibers, and in particular a stretchable non-woven laminate material.

In a particular embodiment, the absorbent pad structure is suspended within the article chassis so as to lie directly against and be slidable relative to the outer cover member even when the article is worn by a user. In other words, the degree of “suspension” of the absorbent pad structure is not so great such that the pad is pulled away from the outer cover member. Thus, it is not necessary to provide a separate liquid impermeable backing sheet against the absorbent core material. Although it may be desired to urge the absorbent pad structure against the wearer, this can be done without pulling the structure away from the outer cover member, which would tend to present a “puffy” and ill-fitting appearance.

An article according to the invention may incorporate any combination of features typically employed in absorbent articles. For example, containment flaps may be disposed alongside of the lateral sides of the absorbent pad structure. Leg elastics may be used to draw the outer cover member against the wearer. The chassis may include partial or full elasticized waistbands. The absorbent article may include various types of side seam configurations. For example, lateral side panel portions may be bonded at the side seams such that the wearer must pull the article on, much like underwear. These bonded seams may also be tearable such that the article may be removed from the wearer by separating or tearing along the seams and removing the article, much like a diaper. In an alternate embodiment, the side seams are releasable and re-attachable. For example, a hook-and-loop or other type of re-attachable system may be used along the side seams.

Aspects of the invention will be described below in greater detail with reference to embodiments shown in the figures.

### BRIEF DESCRIPTION OF THE FIGURES

FIG. 1 is a perspective view of an embodiment of an absorbent article, in this case a child's training pant, with a portion shown in partial cut-away view.

FIG. 2 is a bodyside plan view of an alternate embodiment of an absorbent article, in this case a disposable diaper, in accordance with the invention with a portion thereof shown in partial cut-away view.

FIG. 3 is a perspective view of an embodiment of an absorbent article, in this case a child's training pant, in accordance with the invention with a portion shown in partial cut-away view.

### DETAILED DESCRIPTION

The invention will now be described in detail with reference to particular embodiments thereof. The embodiments are provided by way of explanation of the invention, and are not meant as a limitation of the invention. For example, features described or illustrated as part of one embodiment may be used with another embodiment to yield still a further embodiment. It is intended that the present invention include these and other modifications and variations as come within the scope and spirit of the invention.

Within the context of the present description, the following terms may have the following meanings:

“Attached” refers to the bonding, joining, adhering, connecting, attaching, or the like, of two elements. Two elements are considered to be attached together when they are bonded directly to one another or indirectly to one another, such as when each is directly attached to an intermediate element.

“Elastomeric” refers to a material or composite which can be elongated by at least 25% of its relaxed length and which will recover, upon release of the applied force, at least 10% of its elongation. It is generally preferred that the elastomeric material or composite be capable of being elongated by at least 100%, more preferably by at least 300%, of it relaxed length and recover at least 50% of its elongation. An elastomeric material is thus stretchable and “stretchable” and “elastomeric” may be used interchangeably.

“Elastic” or “Elasticized” means that property of a material or composite by virtue of which it tends to recover or retract towards its original size and shape after removal of a force causing a deformation.

“Neck-bonded” laminate refers to a composite material having an elastic member that is bonded to a non-elastic member while the non-elastomeric member is extended in the machine direction creating a necked material that is elastic in the cross-direction. Examples of neck-bonded laminates are disclosed in U.S. Pat. Nos. 4,965,122; 4,981,747; 5,226,992; and 5,336,545, which are incorporated herein by reference in their entirety for all purposes.

“Stretch-bonded” laminate refers to a composite material having at least two layers in which one layer is a gatherable layer and the other layer is an elastic layer. The layers are joined together when the elastic layer is in an extended condition so that upon relaxing the layers, the gatherable layer is gathered. For example, one elastic member can be bonded to another member while the elastic member is extended at least about 25% of its relaxed length. Such a multiplayer composite elastic material may be stretched until the non-elastic layer is fully extended. Examples of stretch-bonded laminates are disclosed, for example, in U.S. Pat. Nos. 4,720,415, 4,789,699, 4,781,966, 4,657,802, 4,655,760, and PCT application WO 01/88245, which are incorporated herein by reference in their entirety for all purposes.

“Member” when used in the singular can refer to a single element or a plurality of elements.

“Thermoplastic” describes a material that softens when exposed to heat and which substantially returns to a nonsoftened condition when cooled to room temperature.

Various aspects and embodiments of the invention will be described in the context of a disposable absorbent article, such as a child's training pant. It is, however, readily apparent that the present invention could also be employed with other articles, including disposable diapers, feminine care articles, incontinence garments, diaper pants, swim pants, and the like. Typically, the disposable articles are intended for limited use and are not intended to be laundered or otherwise cleaned for reuse. A child's training pant, for example, is discarded after it has become soiled by the wearer.

In its various aspects, the invention can provide a distinctive absorbent article which exhibits desirable physical properties, such as softness, flexibility, conformance, trim appearance, reduced gapping and reduced leakage. The article may include an absorbent body structure that is essentially suspended by at least one end to a waistband structure of the article chassis. In a particular configuration, the absorbent body structure is suspended at both ends. The various configurations according to the invention allow for suspending the structure within the outer cover member and bodyside liner such that the pad structure does not inhibit the desired stretchability of the outer cover and/or bodyside liner. The suspension configuration does not detract from a slim and form-fitting appearance of the article.

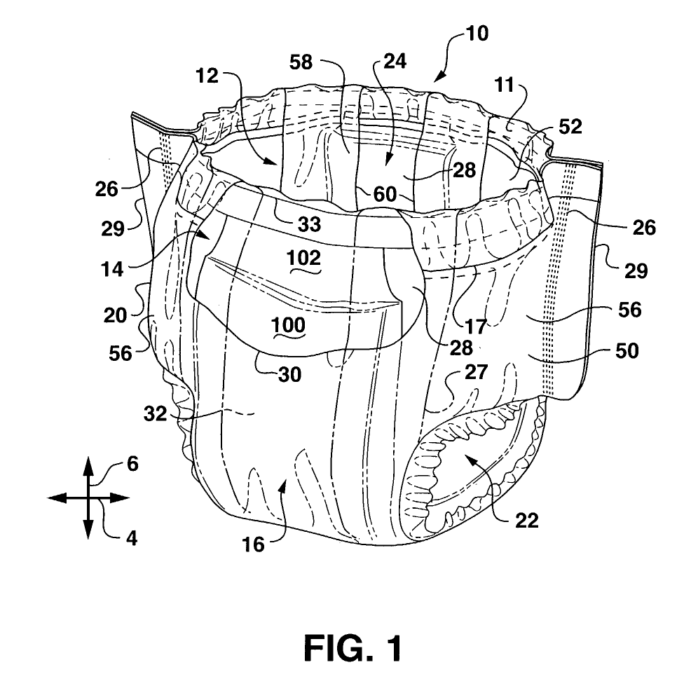

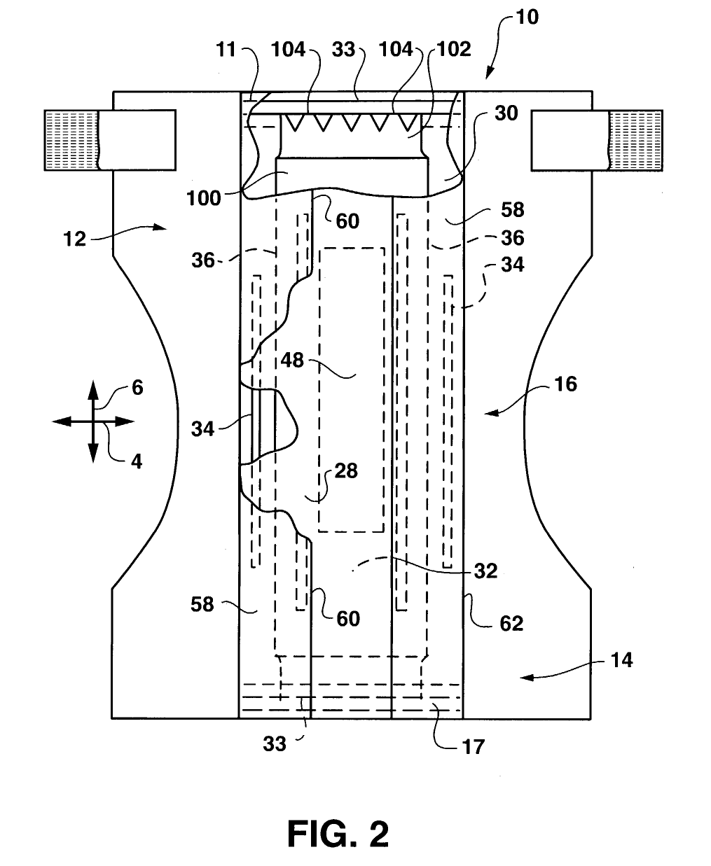

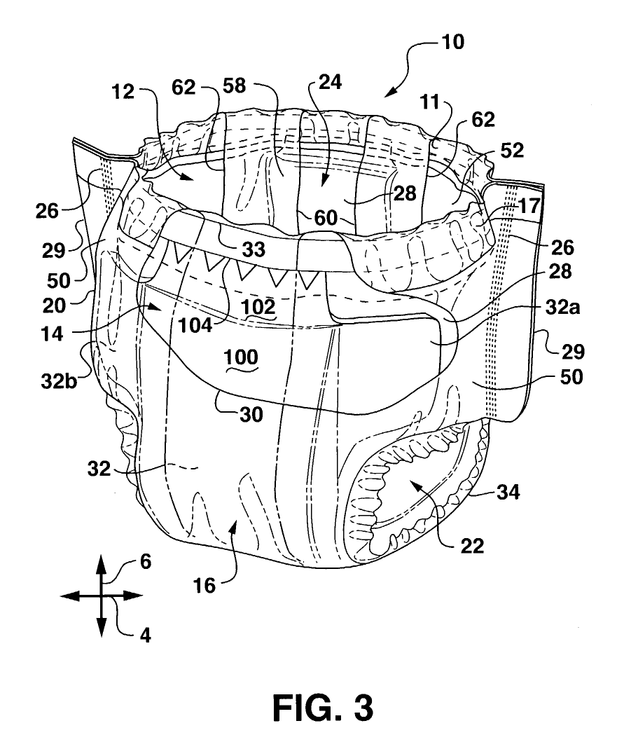

With reference to the figures, an article 10, such as the representatively shown child's training pant in FIGS. 1 and 3 and disposable diaper in FIG. 2, includes a body or chassis 20 having a lengthwise, longitudinal direction 6, a lateral or transverse direction 4, a front waist region 14, a back waist region 12, and an intermediate crotch region 16 interconnecting the front and back waist regions. The waist regions 12 and 14 comprise those portions of the article 10 which when worn, wholly or partially cover or encircle the waist or mid-lower torso of the wearer. In particular configurations, the front 14 and back 12 waist regions may include elastic front and back waistband portions 17,11 incorporating strands of elastic material 33. In the embodiment of FIGS. 1 and 3, the elastic waistband portions 11,17 are generally continuous around a waist opening 24 of the article. In the embodiment of FIG. 2, the waistband portions 11,17 extend only partially across their respective waist regions. The intermediate crotch region 16 lies between and interconnects the waist regions 14 and 12, and comprises that portion of the article 10 which, when worn, is positioned between the legs of the wearer and covers the lower torso of the wearer. Thus, the intermediate crotch region 16 is an area where repeated fluid surges typically occur in the training pant or other disposable absorbent article.

The article 10 includes a substantially liquid-impermeable outer cover member 30, a liquid-permeable bodyside liner 28, and an absorbent body structure 32 sandwiched between the outer cover member 30 and the bodyside liner layer 28. In certain configurations, the outer cover member 30 and absorbent body structure 32 may be stretchable, particularly in the lateral or transverse direction 4. For example, the outer cover member 30 may be made substantially of an elastomeric material, and the absorbent body structure may include an elastomeric absorbent material. In certain embodiments, the bodyside liner 28 is also stretchable. For example, the bodyside liner 28 may be made of an elastomeric material.

In the illustrated embodiment of the article 10, the chassis 20 includes laterally extending front panel portions 50 and back panel portions 52. This configuration is common for training pants. In the embodiment of FIG. 3, the panel portions 50, 52 are an extension of the chassis 20 and may be, for example, extensions of the outer cover member 30, bodyside liner 28, or both. This configuration may be desirable particularly if the chassis 20 is stretchable in the transverse direction 4, particularly across the front and back waist regions 14, 12. This embodiment may also accommodate laterally extending wing portions 32a and 32b of the absorbent body structure 32 between the materials of the panel portions 50, 52, as illustrated in FIG. 3. In particular embodiments, the chassis 20 may include any combination of elastomeric cover member 30, elastomeric bodyside liner 28, and any other elastomeric components that in combination render a stretchable unitary chassis 20.

In an alternate embodiment as illustrated in FIG. 1, the panel portions 50, 52 are defined by generally elastomeric side panels 56 that are attached to lateral sides of the chassis 20, for example along seam lines 27. In this embodiment, the other components of the central structure of the chassis 20 may or may not be elastomeric. A configuration such as this is known from the HUGGIES® PULL-UPS® disposable training pants from Kimberly-Clark Corp. of Neenah, Wis., USA.

The training pant 10 may be of a style and configuration wherein the front and back panel portions 50, 52 have lateral sides 29 that are brought together upon folding the chassis to form a pant structure having the waist opening 24 and leg openings 22. The lateral sides 29 are bonded in a known manner so as to define side seams 26 of the pant structure. With this type of configuration, the pant 10 is pulled on by the wearer in a manner similar to underwear. Desirably, these seams 26 may be separable or tearable so that the pant 10 may be removed from the wearer by tearing at or along the seams 26 and removing the article in a manner similar to a diaper. In an alternate embodiment, the front and back panel portions 50, 52 may be separable and re-attachable at the side seams 26. A fastening system, such as a hook-and-loop system, may be used to interconnect the back waist region 12 with the front waist region 14 to define the pant structure and hold the article on a wearer. Additional suitable releasable fastening systems are described in U.S. Pat. No. 6,231,557 B1 and the International Application WO 00/35395, these references being incorporated herein by reference in their entirety for all purposes.

The article 10 may also incorporate longitudinally extending containment flaps 58 disposed over the bodyside liner 28. The containment flaps 58 have longitudinal ends that are attached to the longitudinal ends of the chassis 20. For example, the flaps 58 may extend over and be attached to the waistband portions 11, 17, for example by ultrasonic bonding. The flaps 58 have an outboard lateral side 62 (FIGS. 2 and 3) that is attached to the chassis 20 desirably outboard of the underlying absorbent body structure 32, although they may also be attached over the absorbent body structure 32. The flaps 58 have a laterally inboard “free” side 60 such that they essentially define a containment pocket along the lateral sides of the absorbent structure 32. Containment flaps 58 as described herein may be of a conventional configuration, such as the flaps used in the HUGGIES® disposable training pants.

FIG. 2 shows a body facing plan view of an alternate embodiment of an article 10 according to the invention, in this case a disposable diaper in its generally flat-out, uncontracted state (i.e., with substantially all elastic induced gathering and contraction removed). As mentioned, the article 10 will typically include a porous, liquid permeable bodyside liner 28; a substantially liquid impermeable outer cover member 30; and an absorbent body structure 32 positioned and attached between the outer cover member and bodyside liner. In certain embodiments, a surge layer 48 may be optionally located adjacent the absorbent structure and attached, for example with an adhesive to the liner 28 and to the absorbent core material. Elastomeric gathering members, such as leg elastics 34 and waist elastics 33 may be provided, as is well known in the art. The waist elastics 33 may be composed of any suitable elastomeric material, such as an elastomeric film, an elastic foam, multiple elastic strands, an elastomeric fabric, and the like. Embodiments of waistband structures that may be utilized with articles 10 according to the invention are also described in U.S. Pat. Nos. 5,601,547; 6,358,350 B1; 6,336,921 B1; and 5,711,832, incorporated by reference in their entirety for all purposes. The liner 28, outer cover 30, absorbent structure 32, surge layer 48, and elastic members 34 and 33 may be assembled together into a variety of well-known absorbent article configurations.

Referring to the cut-away portions of FIGS. 1-3, the absorbent pad structure 32 is sandwiched between the outer cover member 30 and the bodyside liner 28, and includes structure comprising an absorbent core coform material 100 having a fusable thermoplastic material. For example, the coform absorbent core 100 may be a cellulosic fibrous air laid mat containing staple length polymer fibers or another type of fusable material that softens or melts to an extent upon heat being applied thereto in a suitable bonding process. Suitable coform materials will be described in greater detail below.

The absorbent pad structures 32 are suspended relative to the chassis 20 by way of at least one suspension member 102 disposed at a longitudinal end of the pad structure 32. The pad structure may be suspended at each of its longitudinal ends with a respective suspension member 102, as illustrated in the figures. The suspension member 102 has an end fusably bonded with the fusable thermoplastic material within the absorbent core material 100. For example, the suspension member may be a nonwoven material, such as a spunbond material, of a fusable thermoplastic polymer material that also melts or softens and fuses with the molten thermoplastic material of the core material 100 in the bonding process. Suitable materials for the suspension members 102 will be described below. The end of the suspension member 102 may be inserted into the core material 100 or may be laid upon a surface of the core material 100. The components are then bonded together by any one of a number of techniques known to those skilled in the art such that the thermoplastic materials become molten and fuse upon subsequent solidification. Thus, the suspension members 102 are attached directly to the absorbent core material 100 without the necessity of a separate attaching layer, such as a facing or backing sheet. The suspension members 102 can easily be incorporated into in-line processing steps for aligning and attaching the outer cover member 30, bodyside liner 28, and elastic waistband structures 11, 17.

The opposite end of the suspension members 102 is attached to a respective waistband structure 11 or 17 by any suitable method, such as adhesive, bonding, and the like. For example, the end of the suspension member 102 may be adhered or bonded in the waistband forming process along with the waist elastic members 33. The suspension members 102 may be bonded to the waistband structures 11, 17 along a generally continuous seam, as indicated in FIG. 1. Alternately, it may be desired to bond the suspension members 102 in a discontinuous manner, such as the spaced-apart bonds 104 generally illustrated in FIGS. 2 and 3. This type of attachment may be beneficial in that the suspension members 102 would not inhibit the stretchability of the elastic waistband portions 11, 17.

In the illustrated embodiment, the suspension members are shown as generally wedge-shaped for illustrative purposes only. It should be appreciated that the suspension members may take on any suitable shape, such as a solid rectangular strip, scalloped segment, notched segment, etc. Any number of shapes are suitable for connecting the absorbent pad structure 32 to the waistband structures 11, 17.

In its suspended attachment configuration, the absorbent pad structure 32 lies sandwiched between and against the outer cover member 30 and bodyside liner 28 such that it is slidable relative to these components. In this manner, the pad structure is not pulled away from the outer cover member 30 and there are no significant gaps or spaces therebetween. Although the suspension members 102 may tend to draw the absorbent pad structure 32 against the wearer, the overall configuration of the chassis 20 is such that the outer cover member 30 is also drawn against the absorbent pad structure.

It may be desired to provide the absorbent pad structure with a degree of longitudinal stretchability. In this regard, the suspension members 102 may be formed of an elastomeric material, such as a stretch-bonded laminate, a neck-bonded laminate, a necked or creped spunbond material, or any other suitable stretchable material known in the art for use in absorbent articles. For the embodiment wherein the suspension members 102 are elastomeric, the absorbent core 100 may be generally non-elastic. Alternately, the absorbent core material 100 may also be elastomeric, for example an elastic coform absorbent material. If the absorbent core material 100 is elastomeric, it may not be necessary for the suspension member 102 to also be elastomeric.

As discussed, the absorbent core material 100 may be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein in their entirety by reference thereto for all purposes.

With particular embodiments of an absorbent article according to the invention, it may be desired that the absorbent core material 100 is elastomeric. For this purpose, the absorbent web material can include elastomeric fibers in an amount which is at least a minimum of about 2 wt %. The amount of elastomeric fibers can alternatively be at least about 3 wt %, and can optionally be at least about 5 wt % to provide improved performance. In addition, the amount of elastomeric fibers can be not more than about 60 wt %. Alternatively, the amount of elastomeric fibers can be not more than about 45 wt %, and optionally, can be not more than about 30 wt % to provide improved benefits. These values are important because they can provide the absorbent retention portion with desired levels of stretchability and structural stability without excessively degrading the physical properties or the liquid-management properties of the absorbent structure. An absorbent web material with an excessively low proportion of elastomeric fibers may be insufficiently stretchable, and a web material with an excessively high proportion of elastomeric fibers may exhibit an excessive degradation of its absorbency functionalities, such as poor intake, poor distribution, poor retention of liquid and/or an excessive tension force when stretched.

The absorbent core material 100 may be an elastomeric coform absorbent web material. In particular aspects, the elastomeric coform material can have an overall coform basis weight which is at least a minimum of about 50 grams per square meter (g/m²). The coform basis weight can alternatively be at least about 100 g/m² and can optionally be at least about 200 g/m² to provide improved performance. In addition, the coform basis weight can be not more than about 1200 g². Alternatively, the coform basis weight can be not more than about 900 g/m², and optionally, can be not more than about 800 g/m² to provide improved benefits. These values are important because they can provide the absorbent body structure with desired stretchability and structural stability without excessively degrading the physical properties or the liquid-management functionalities of the absorbent body structure. Retention portions having excessively low proportions of elastomeric coform material may not be sufficiently stretchable. An absorbent web material having excessively large amounts of elastomeric coform materials can exhibit an excessive degradation of their absorbency functionalities, such as an excessive degradation of intake, distribution and/or retention properties.

Other examples of elastomeric absorbent structures are described in U.S. Pat. No. 6,362,389 B1, incorporated herein by reference for all purposes.

The absorbent core material 100 utilized in the absorbent body structure 32 is also selected so that the individual absorbent body structure possesses a particular individual total absorbency depending on the intended article of use. For example, for infant care products, the total absorbency can be within the range of about 200-900 grams of 0.9 wt % saline, and can typically be about 500 g of saline. For adult care products, the total absorbency can be within the range of about 400-2000 grams of saline, and can typically be about 1300 g of saline. For feminine care products, the total absorbency can be within the range of about 7-50 grams of menstrual fluid, and can typically be within the range of about 30-40 g of menstrual fluid.

As a general rule, superabsorbent material is present in the absorbent web in an amount of from about 0 to about 90 weight percent based on total weight of the web. The web may have a density within the range of about 0.10 to about 0.35 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a suberabsorbent material is capable of absorbing at least about 15 times its weight in liquid, and desirably is capable of absorbing more than about 25 times its weight in liquid. Suitable superabsorbent materials are readily available from various suppliers. For example, Favor 880 superabsorbent is available from Stockhausen GmbH of Germany; and Drytech 2035 is available from Dow Chemical Company, of Midland Mich., USA.

As mentioned in the discussion of FIG. 2, the absorbent body structure 32 may also include a surge management layer 48 which helps to decelerate and diffuse surges or gushes of liquid that may be rapidly introduced into the absorbent body of the article. Desirably, the surge management layer can rapidly accept and temporarily hold the liquid prior to releasing the liquid into the storage or retention portions of the absorbent structure. The surge layer can be located below the bodyside liner layer 28. Alternatively, the surge layer may be located on the body facing surface of the bodyside liner 28. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference in their entirety for all purposes.

The suspension members 102 may be formed from a wide variety of materials. The members 102 may, for example, be composed of a single layer, multiple layers, laminates, spunbond fabrics, films, meltblown fabrics, elastic netting, microporous web, bonded carded webs or foams comprised of elastomeric or polymeric materials. Elastomeric nonwoven laminate webs may include a nonwoven material joined to one or more gatherable nonwoven webs, films, or foams. Stretch Bonded Laminates (SBL) and Neck Bonded Laminates (NBL) are examples of elastomeric composites. Nonwoven fabrics are any web of material which has been formed without the use of textile weaving processes which produce a structure of individual fibers which are interwoven in an identifiable repeating manner. Examples of suitable materials are Spunbond-Meltblown fabrics, Spunbond-Meltblown-Spunbond fabrics, Spunbond fabrics, or laminates of such fabrics with films, foams, elastic strand/non-woven laminates, or other nonwoven webs. Elastomeric materials may include cast or blown films, foams, or meltblown fabrics composed of polyethylene, polypropylene, or polyolefin copolymers, as well as combinations thereof. The suspension members 102 may include materials that have elastomeric properties through a mechanical process, printing process, heating process, or chemical treatment. For examples such materials may be apertured, creped, neck-stretched, heat activated, embossed, and micro-strained; and may be in the form of films, webs, and laminates.

The bodyside liner 28 and outer cover member 30 may be generally coextensive, and may have length and width dimensions which are generally larger than and extend beyond the corresponding dimensions of the absorbent structure 32 to provide for the corresponding side and end margins. Optionally, the bodyside liner 28 and outer cover member 30 may not be coextensive. The outer cover member 30 may be composed of a liquid permeable material, but desirably comprises a material which is configured to be substantially impermeable to liquids. For example, a typical outer cover can be manufactured from a thin plastic film, a composite laminate, or other flexible, substantially liquid-impermeable material. As used in the present specification, the term “flexible” refers to materials which are compliant and which will readily conform to the general shape and contours of the wearer's body.

Alternative constructions of the outer cover member 30 may comprise a woven or non-woven fibrous web layer which has been totally or partially constructed or treated to impart the desired levels of liquid impermeability to selected regions that are adjacent or proximate the absorbent body. For example, the outer cover may include a gas-permeable, nonwoven fabric layer laminated to a polymer film layer which may or may not be gas-permeable. Other examples of fibrous, cloth-like outer cover materials can comprise a stretch thinned or stretch thermal laminate material. Although the outer cover 30 typically provides the outermost layer of the article, optionally the article may include a separate outer cover component member which is additional to the outer cover member.

In a particular embodiment, the outer cover 30 may be formed substantially from an elastomeric material and is thus stretchable. In this embodiment, the outer cover 30 may define the entire front and back waist regions 14, 12, including the front and back panel portions 50, 52. The outer cover 30 may, for example, be composed of a single layer, multiple layers, laminates, spunbond fabrics, films, meltblown fabrics, elastic netting, microporous web, bonded carded webs or foams comprised of elastomeric or polymeric materials. The outer cover 30 may be an elastomeric nonwoven laminate web, as described above. Examples of suitable materials are Spunbond-Meltblown fabrics, Spunbond-Meltblown-Spunbond fabrics, Spunbond fabrics, or laminates of such fabrics with films, foams, or other nonwoven webs. Elastomeric materials may include cast or blown films, foams, or meltblown fabrics composed of polyethylene, polypropylene, or polyolefin copolymers, as well as combinations thereof. The elastomeric materials may include polyether block amides such as PEBAX® elastomer (available from AtoChem located in Philadelphia, Pa.), HYTREL® elastomeric polyester (available from E. I. DuPont-de Nemours located in Wilmington, Del.), KRATON® elastomer (available from Shell Chemical Company located in Houston, Tex.), or strands of LYCRA® elastomer (available from E. I. DuPont de Nemours located in Wilmington, Del.), or the like, as well as combinations thereof. The outer cover 30 may include materials that have elastomeric properties through a mechanical process, printing process, heating process, or chemical treatment. For examples such materials may be apertured, creped, neck-stretched, heat activated, embossed, and micro-strained; and may be in the form of films, webs, and laminates.

In an alternate embodiment, the chassis 20 may be substantially non-elastomeric along the crotch region 16. In this embodiment, the outer cover 30 may be made of a generally non-elastomeric material and essentially covers only the crotch region 16. As discussed, the elastomeric side panels 56 provide a desired degree of stretchability to the chassis 20, particularly around the waist and sides of the wearer. A structure of this type is known, for example, from the HUGGIES® PULL-UPS® disposable training pants. The pair of transversely opposed side panels 56 may be permanently bonded to the lateral sides of the chassis structure at bond lines 27 (FIG. 1) using attachment means known to those skilled in the art, such as adhesive, thermal or ultrasonic bonding. Particular examples of suitable constructions for securing a pair of elastically stretchable members to the lateral, side portions of an article to extend laterally outward beyond the laterally opposed side regions of the outer cover and liner components of an article can be found in U.S. Pat. No. 4,938,753, which is incorporated by reference herein in its entirety for all purposes. The lateral outboard sides of the side panels 56 may then be permanently or releasably attached along side seams 26 to define a pant structure. These bonded side seams may be tearable as discussed above.

Suitable elastic materials for the side panels 56 (which may also be suitable for the suspension members 102), as well as a described process of incorporating elastic side panels into a training pant, are described, for example, in the following U.S. Pat. Nos. 4,940,464; 5,224,405; 5,104,116; 5,046,272; and WO 01/88245, all of which are incorporated herein by reference in their entirety for all purposes. In particular embodiments, the elastic material comprises a stretch-thermal laminate (STL), a neck-bonded laminate (NBL), a reversibly necked laminate, or a stretch-bonded laminate (SBL) material. Methods of making such materials are described, for example, in U.S. Pat. Nos. 4,663,220; 5,226,992; and the EP Application 0 217 032, all of which are incorporated herein by reference in their entirety for all purposes.

The bodyside liner 28 presents a body-facing surface which is compliant, soft-feeling, and non-irritating to the wearer's skin. Further, the bodyside liner 28 can be less hydrophilic than the absorbent body 32, and is sufficiently porous to be liquid permeable, permitting liquid to readily penetrate through its thickness to reach the absorbent body. A suitable bodyside liner layer 28 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner layer 28 is typically employed to help isolate the wearer's skin from liquids held in absorbent body 32.

Various woven and nonwoven fabrics can be used for bodyside liner 28. For example, the bodyside liner may include a meltblown web, a spunbonded web, or a bonded-carded-web composed of the desired fibers. The various fabrics can be composed of natural fibers, synthetic fibers or combinations thereof. In particular aspects, the bodyside liner 28 may be comprised of polymer fibers, networks, laminates, liquid permeable films, cellulosic fibers, rayon, water swellable gels, and elastomeric materials, as well as combinations thereof. Suitable materials for the bodyside liner can include meltblown webs, airlaid webs, spunbond webs, or bonded-carded webs of synthetic continuous or discrete polymer fibers and/or natural fibers, a pattern bonded spunbonded web, airlaid web, or bonded carded web, as well as combinations thereof. Suitable polymers can include polypropylene, polyethylene, polyester, and bicomponent materials composed of these polyolefins.

The bodyside liner fabrics may be composed of a substantially hydrophobic material, and the hydrophobic material may optionally be treated with a surfactant or otherwise processed to impart a desired level of wettability and hydrophilicity. In a particular embodiment of the invention, the bodyside liner 28 can be a nonwoven, spunbond polypropylene fabric which is necked approximately 60%. Strands of KRATON® G2760 elastomer material may be adhered to the necked spunbond material. The fabric can be surface treated with an operative amount of surfactant, such as about 0.6% AHCOVEL Base N62 surfactant, available from ICI Americas, a business having offices located in Wilmington, Del. The surfactant can be applied by any conventional means, such as spraying, printing, brush coating or the like.

In particular embodiments wherein it is desired that the bodyside liner layer 28 be stretchable, suitable elastomeric materials can include elastic strands, LYCRA® elastics, elastic films, cast or blown; nonwoven elastic webs, meltblown or spunbond elastomeric fibrous webs, as well as combinations thereof. Examples of elastomeric materials include KRATON® elastomers, HYTREL® elastomers, ESTANE® elastomeric polyurethanes (available from B.F. Goodrich and Company located in Cleveland, Ohio), or PEBAX® elastomers. The bodyside liner may include blends or laminates of fibers, scrim, webs, and films with perforations, apertures, creping, heat activation, embossing, micro-straining, chemically treatment, or the like, as well as combinations thereof.

The bodyside liner 28 and outer cover 30 are connected or otherwise associated together in an operable manner. As used herein, the term “associated” encompasses configurations in which the bodyside liner 28 is directly joined to the outer cover 30 by affixing the bodyside liner 28 directly to the outer cover 30, and configurations wherein the bodyside liner 28 is indirectly joined to the outer cover 30 by affixing the bodyside liner 28 to intermediate members which in turn are affixed to the outer cover 30. The bodyside liner 28 and outer cover 30 can, for example, be joined to each other in at least a portion of the training pant periphery by suitable attachment mechanisms (not shown) such as adhesive bonds, ultrasonic bonds, thermal bonds, pinning, stitching or any other attachment technique known in the art, as well as combinations thereof. For example, a uniform continuous layer of adhesive, a patterned layer of adhesive, a sprayed pattern of adhesive or an array of separate lines, swirls or spots of construction adhesive may be used to affix the bodyside liner 28 to the outer cover 30. It should be readily appreciated that the above-described attachment means may also be employed to suitably interconnect, assemble and/or affix together the various other component parts of the articles which are described herein.

It should be understood that resort may be had to various other embodiments, modifications, and equivalents to the embodiments of the invention described herein which, after reading the description of the invention herein, may suggest themselves to those skilled in the art without departing from the scope and spirit of the present invention.

What is claimed is:

1. An absorbent article, comprising:
a chassis having a front waist region with a front waistband, a back waist region with a back waistband, and a crotch region extending longitudinally between said front and back waist regions;
a generally liquid impermeable and stretchable outer cover member, and a bodyside liner extending from said front waist band to said back waist band;
an absorbent pad structure sandwiched between said outer cover member and said bodyside liner, said absorbent pad structure comprising an absorbent core coform material having a fusable thermoplastic material;
a suspension member disposed at each longitudinal end of said absorbent pad structure, said suspension members having one end fusably bonded with said fusable thermoplastic material within said absorbent core material, and an opposite end attached to a respective said waistband of said chassis; and
wherein upon said article being worn by a user, said absorbent pad structure is slideably disposed against said outer cover member such that said absorbent article is form-fitting without said absorbent pad structure impeding stretchability of said outer cover member.

2. The article as in claim 1, wherein said suspension members are formed of an elastomeric material.

3. The article as in claim 2, wherein said coform material is generally non-elastomeric.

4. The article as in claim 1, wherein said suspension members are generally non-elastomeric.

5. The article as in claim 4, wherein said coform material is generally elastomeric.

6. The article as in claim 5, wherein said fusable thermoplastic material comprises elastic thermoplastic fibers.

7. The article as in claim 1, wherein said coform material is a generally elastic coform material and said suspension members are formed of an elastomeric material.

8. The article as in claim 1, wherein said suspension members are ultrasonically bonded with said fusable thermoplastic material within said absorbent core material.

9. The article as in claim 8, wherein said suspension members comprise an elastomeric non-woven material.

10. An absorbent article, comprising:
a chassis having a front waist region with a front waistband, a back waist region with a back waistband, and a crotch region extending longitudinally between said front and back waist regions;
a generally liquid impermeable outer cover member and a generally coextensive bodyside liner;
a longitudinally disposed absorbent pad structure sandwiched between said outer cover member and said bodyside liner, said absorbent pad structure comprising an absorbent core material having a fusable thermoplastic material;
at least one elastic suspension member disposed at a longitudinal end of said absorbent pad structure, said suspension member having one end fusably bonded with said fusable thermoplastic material within said absorbent core material, and an opposite end attached at a respective said waistband of said chassis.

11. The article as in claim 10, wherein said absorbent core material comprises an elastic coform material.

12. The article as in claim 10, wherein said absorbent core material comprises a generally inelastic coform material.

13. The article as in claim 10, wherein said suspension member is bonded between said outer cover member and said bodyside liner at said respective waistband.

14. The article as in claim 10, further comprising longitudinally extending containment flaps, said flaps having a laterally outward side attached to said chassis generally outboard of said absorbent pad structure and an opposite free laterally inboard side extending over said bodyside liner so as to form containment pockets along the longitudinal sides of said absorbent pad structure.

15. The article as in claim 14, wherein said laterally inboard sides are elasticized.

16. The article as in claim 10, wherein said outer cover member is an elastomeric material.

17. The article as in claim 10, wherein said suspension member comprise a fusable thermoplastic material that fuses with said thermoplastic material of said absorbent core material.

18. The article as in claim 10, comprising a respective said suspension member at each longitudinal end of said absorbent pad structure.
